(19) United States
(12) Patent Application Publication
(10) Pub. No.: US 2026/0038651 A1
(43) Pub. Date: Feb. 5, 2026

(54) PHARMACY-TRANSACTION APPARATUSES AND RELATED METHODS

(71) Applicant: MEGA PHARM LIMITED, Calgary (CA)

(72) Inventor: Osakpamwan UWUBANMWEN, Calgary (CA)

(21) Appl. No.: 18/994,279

(22) PCT Filed: Jul. 14, 2023
(86) PCT No.: PCT/CA2023/050950

§ 371 Date: Jan. 14, 2025

Related U.S. Application Data
(60) Provisional application No. 63/389,475, filed on Jul. 15, 2022.

(51) Int. Cl. G16H 10/60; A47F 10/00; G16H 20/13; G16H 40/20

(52) U.S. Cl. CPC G16H 10/60; A47F 10/00; G16H 20/13; G16H 40/20

(57) ABSTRACT

A pharmacy-transaction apparatus includes at least one booth, each booth of the at least one booth accessible from at least a customer side of a pharmacy counter and a dispensing side of the pharmacy counter opposite the customer side of the pharmacy counter, wherein, at least, at least one pharmacy-transaction device is in one or more of the at least one booth.

### CROSS-REFERENCE TO RELATED APPLICATION

This application claims the benefit of, and priority to, U.S. provisional patent application No. 63/389,475 filed Jul. 15, 2022. U.S. provisional patent application No. 63/389,475 is incorporated by reference herein.

### FIELD

This disclosure relates generally to pharmacy-transaction apparatuses and related methods.

### RELATED ART

Pharmacy transactions can involve discussing confidential information without adequate privacy.

### SUMMARY

According to at least one embodiment, there is disclosed a pharmacy-transaction apparatus comprising at least one booth, each booth of the at least one booth accessible from at least a customer side of a pharmacy counter and a dispensing side of the pharmacy counter opposite the customer side of the pharmacy counter, wherein, at least, at least one pharmacy-transaction device is in one or more of the at least one booth.

In some embodiments, one or more of the at least one booth is adjacent the pharmacy counter.

In some embodiments, one or more of the at least one booth is integrated into the pharmacy counter.

In some embodiments, one or more of the at least one booth is attached to the pharmacy counter.

In some embodiments, one or more of the at least one booth is attached directly to the pharmacy counter.

In some embodiments, at least portions of the pharmacy counter are on opposite sides of one or more of the at least one booth.

In some embodiments, the apparatus further comprises the pharmacy counter.

In some embodiments, the pharmacy counter comprises retail fixtures for displaying pharmacy products on the customer side of the pharmacy counter.

In some embodiments, the pharmacy counter comprises retail fixtures displaying pharmacy products on the customer side of the pharmacy counter.

In some embodiments, at least some of the retail fixtures are on opposite sides of one or more of the at least one booth.

In some embodiments, at least some of the retail fixtures are shelves.

In some embodiments, at least some of the retail fixtures are racks.

In some embodiments, the pharmacy counter comprises product-storage fixtures for storing pharmacy products on the dispensing side of the pharmacy counter.

In some embodiments, the pharmacy counter comprises product-storage fixtures storing pharmacy products on the dispensing side of the pharmacy counter.

In some embodiments, at least some of the product-storage fixtures are on opposite sides of one or more of the at least one booth.

In some embodiments, at least some of the product-storage fixtures are shelves.

In some embodiments, at least some of the product-storage fixtures are racks.

In some embodiments, each booth of the at least one booth is substantially enclosed on all sides.

In some embodiments, each booth of the at least one booth is enclosed on all sides.

In some embodiments, the apparatus further comprises the at least one pharmacy-transaction device.

In some embodiments, the at least one pharmacy-transaction device comprises at least one payment device.

In some embodiments, the at least one pharmacy-transaction device comprises at least one pharmacy computer.

In some embodiments, the at least one pharmacy computer is configured to access at least a database of customer medication records.

In some embodiments, one or more of the at least one booth comprises at least one translucent exterior wall.

In some embodiments, one or more of the at least one booth comprises at least one translucent exterior wall on each side of the booth.

In some embodiments, the at least one translucent exterior wall permits visibility from within the one or more of the at least one booth to at least substantially all of a customer area on the customer side of the pharmacy counter.

In some embodiments, the apparatus further comprises at least one air pump in communication with an interior of one or more of the at least one booth and with an exterior of the at least one booth, the at least one air pump operable to exchange air between the interior of the one or more of the at least one booth and the exterior of the at least one booth.

In some embodiments, the apparatus further comprises lighting associated with one or more of the at least one booth operable to illuminate an interior of the one or more of the at least one booth.

In some embodiments, one or more of the at least one booth is wheelchair-accessible from at least the customer side of the pharmacy counter.

In some embodiments, one or more of the at least one booth is wheelchair-accessible from at least the dispensing side of the pharmacy counter.

In some embodiments, one or more of the at least one booth has a sound transmission class (STC) rating of at least 40.

In some embodiments, one or more of the at least one booth has an STC rating of at least 45.

In some embodiments, one or more of the at least one booth has an STC rating of at least 50.

In some embodiments, one or more of the at least one booth has an STC rating of at least 55.

In some embodiments, one or more of the at least one booth has an STC rating of at least 60.

In some embodiments, the at least one booth comprises a first booth and a second booth.

In some embodiments, each booth of the at least one booth is sufficiently soundproof to permit confidential discussions in each booth of the at least one booth with at least auditory privacy.

In some embodiments, each booth of the at least one booth is sufficiently soundproof to permit collection or disclosure of personal information in each booth of the at least one booth with at least auditory privacy.

In some embodiments, each booth of the at least one booth sufficiently diffuses light exiting, or sufficiently blocks light from exiting, each booth of the at least one booth to permit confidential transactions in each booth of the at least one booth with at least visual privacy.

In some embodiments, each booth of the at least one booth sufficiently diffuses light exiting, or sufficiently blocks light from exiting, each booth of the at least one booth to permit confidential transactions in each booth of the at least one booth with at least visual privacy.

According to at least one embodiment, there is disclosed a method of conducting at least one pharmacy transaction with a pharmacy customer, the method comprising conducting the at least one pharmacy transaction in one or more of the at least one booth.

In some embodiments, the at least one pharmacy transaction is at least one confidential pharmacy transaction.

In some embodiments, the at least one pharmacy transaction comprises receiving at least one prescription from the pharmacy customer.

In some embodiments, the at least one pharmacy transaction comprises providing at least one medication to the pharmacy customer.

In some embodiments, the at least one pharmacy transaction comprises: receiving at least one prescription from the pharmacy customer in the first booth; and providing at least one medication to the pharmacy customer in the second booth.

In some embodiments, the at least one pharmacy transaction comprises receiving a payment from the pharmacy customer.

In some embodiments, receiving the payment from the pharmacy customer comprises receiving the payment from the pharmacy customer using the at least one payment device.

In some embodiments, the at least one pharmacy transaction comprises providing pharmaceutical advice to the pharmacy customer.

In some embodiments, the at least one pharmacy transaction comprises causing the at least one pharmacy computer to access at least the database of customer medication records.

In some embodiments, the at least one pharmacy transaction comprises receiving personal information from the pharmacy customer.

In some embodiments, the at least one pharmacy transaction comprises causing the at least one pharmacy computer to cause at least the database of customer medication records to store at least some of the personal information.

Other aspects and features will become apparent to those ordinarily skilled in the art upon review of the following description of illustrative embodiments in conjunction with the accompanying figures.

### BRIEF DESCRIPTION OF THE DRAWINGS

FIG. 1 is a plan view of a pharmacy according to one embodiment.

FIG. 2 is a perspective view of a booth of the pharmacy of FIG. 1.

FIG. 3 is a perspective view of a booth according to another embodiment.

FIG. 4 is a schematic illustration of a pharmacy-transaction system including pharmacy-transaction devices in the booth of FIG. 2.

### DETAILED DESCRIPTION

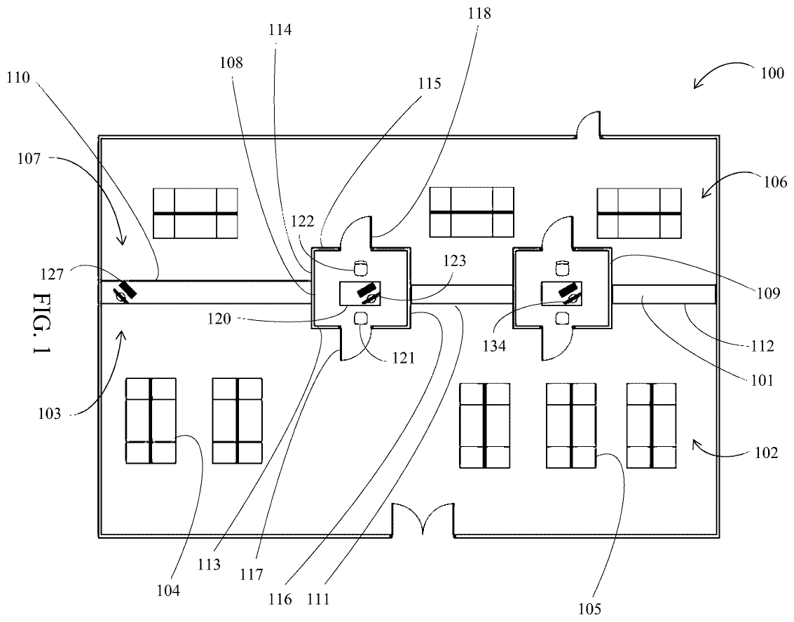

Referring to FIG. 1, a pharmacy according to one embodiment is shown generally at 100. The pharmacy 100 includes a pharmacy counter 101. A customer area of the pharmacy 100 is shown generally at 102 and is on a customer side shown generally at 103 of the pharmacy counter 101. The customer area 102 may be accessible by the general public or by others (such as hospital patients, care-home residents, or other groups of individuals, for example). The customer area 102 may include retail fixtures (such as retail fixtures 104 and 105, for example) for displaying, or actually displaying, pharmacy products (such as medications, medical devices, one or more other pharmacy products, or a combination of two or more thereof) in the customer area 102. Such retail fixtures may include one or more shelves, one or more racks, one or more other retail fixtures, or a combination of two or more thereof, and may be spaced apart from the pharmacy counter 101 as shown in FIG. 1 or alternatively adjacent or attached to the pharmacy counter 101

A dispensing area of the pharmacy 100 is shown generally at 106 and is on a dispensing side shown generally at 107 of the pharmacy counter 101 and opposite the customer side 103 of the pharmacy counter 101. In general, the dispensing area 106 includes stored medication (such as medication that may require a prescription, for example) and other pharmacy equipment (such as dispensing or compounding equipment, for example). Also, in general, the dispensing area 106 is accessible to pharmacists, pharmacy technicians, and other pharmacy staff, and access to the dispensing area 106 by the general public is restricted.

The pharmacy counter 101 may function as a barrier to restrict access between the customer area 102 and the dispensing area 106. For example, the pharmacy counter 101 may have a height and width between the customer area 102 and the dispensing area 106 that obstructs or prevents individuals from passing between the customer area 102 and the dispensing area 106. However, the pharmacy counter 101 is an example only, and alternative embodiments may differ. For example, alternative embodiments may include one or more alternatives to the pharmacy counter 101.

The pharmacy 100 also includes a booth 108 and a booth 109, although alternative embodiments may include only a single booth or more than two booths. The booth 108, the booth 109, or both may be adjacent the pharmacy counter 101, integrated into the pharmacy counter 101, attached indirectly to the pharmacy counter 101, attached directly to the pharmacy counter 101, or a combination of two or more thereof. Portions of the pharmacy counter 101 are on opposite sides of the booths 108 and 109. For example, in the embodiment shown, the pharmacy counter 101 includes portions 110, 111, and 112. The portions 110 and 111 are on opposite sides of the booth 108, and the portions 111 and 112 are on opposite sides of the booth 109. However, alternative embodiments may differ. For example, in alternative embodiments a pharmacy counter may be spaced apart from a booth or may be on only one side of a booth.

The pharmacy counter 101 may include retail fixtures for displaying, or actually displaying, pharmacy products (such as medications, medical devices, one or more other pharmacy products, or a combination of two or more thereof) on the customer side 103 of the pharmacy counter 101. Such retail fixtures may be on opposite sides of the booth 108, on opposite sides of the booth 109, or both. Such retail fixtures may include one or more shelves, one or more racks, one or more other retail fixtures, or a combination of two or more thereof. However, alternative embodiments may differ, and alternative embodiments may include other retail fixtures or may omit retail fixtures from the pharmacy counter 101.

Also, the pharmacy counter 101 may include product-storage fixtures for storing, or actually storing, pharmacy products (such as medications, medical devices, one or more other pharmacy products, or a combination of two or more thereof) on the dispensing side 107 of the pharmacy counter 101. Such product-storage fixtures may be on opposite sides of the booth 108, on opposite sides of the booth 109, or both. Such product-storage fixtures may include one or more shelves, one or more racks, one or more other product-storage fixtures, or a combination of two or more thereof. However, alternative embodiments may differ, and alternative embodiments may include other product-storage fixtures or may omit product-storage fixtures from the pharmacy counter 101.

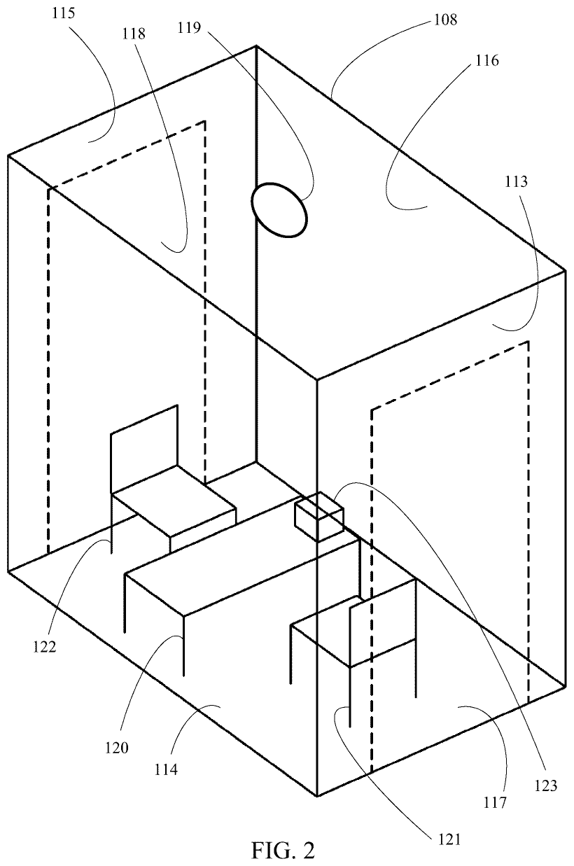

As shown in FIG. 2, the booth 108 has side walls 113, 114, 115, and 116. The booth 108 may have a square, rectangular, circular, or other shape. The booth 108 may have length, width, and other dimensions suitable for up to four individuals in the booth 108.

The booth 108 includes a door 117 in the wall 113 and a door 118 in the wall 115. The door 117, when open, permits access between an interior of the booth 108 and the customer side 103. The door 118, when open, permits access between the interior of the booth 108 and the dispensing side 107. The booth 108 is therefore accessible from at least the customer side 103 of the pharmacy counter 101 and the dispensing side 107 of the pharmacy counter 101. The booth 108 may be wheelchair-accessible from the customer side 103 of the pharmacy counter 101, from the dispensing side 107 of the pharmacy counter 101, or both. The walls 113, 114, 115, and 116 and the doors 117 and 118 may individually or collectively be referred to as exterior walls of the booth 108.

In some embodiments, one or both of doors 117 and 118 may be lockable by any suitable locking mechanism. For example, a customer may be able to lock door 117 from the interior of booth 108 to prevent another customer entering the booth. Further, when booth 108 is not use (such as when a pharmacist is not present), by locking one of doors 117 and 118 access to the dispensing side 107 from the customer side 103 may be prevented.

The booth 108 may be constructed from any suitable materials. For example, portions of booth 108 may be constructed from wood, metal, glass, plastic, or fabric (such as cotton).

In the embodiment shown, the walls 113, 114, 115, and 116 and the doors 117 and 118 include, consist essentially of, or consist of translucent plastic, translucent glass, one or more other translucent materials, or a combination of two or more thereof. Herein, “translucent” includes but is not necessarily limited to “transparent”. As a result, the booth 108 has at least one translucent exterior wall, and at least one translucent exterior wall on each side (namely the sides of the walls 113, 114, 115, and 116 and the doors 117 and 118) of the booth 108. Further, the walls 113, 114, 115, and 116 and the doors 117 and 118 permit visibility from within the booth 108 to at least substantially all of the customer area 102. Such visibility may allow a pharmacist, pharmacy technician, or other pharmacy staff to conduct a pharmacy transaction in the booth 108 while working alone. In some embodiments, one or more exterior walls of the booth 108 may allow a pharmacist, pharmacy technician, or other pharmacy staff to see at least substantially all of the customer area 102 from within the booth 108 while preventing other individuals in customer area 102 from seeing a customer in the booth 108.

Further, lighting may be in the booth 108, or lighting in the booth 108 may be provided from lighting exterior to the booth 108. In the embodiment shown, a light source 119 is associated with the booth 108 and operable to illuminate an interior of the booth 108. The light source 119 may include one or more incandescent light sources, one or more fluorescent light sources, one or more light-emitting diodes (LEDs), one or more other light sources, or a combination of two or more thereof. However, alternative embodiments may differ, and in other embodiments, lighting for an interior of the booth 108 may be provided by one or more light sources that may be exterior to, interior to, or part of the booth 108.

In some embodiments, one or more exterior walls of the booth 108 may allow a pharmacist, pharmacy technician, other pharmacy staff and/or a customer to see within the booth 108 from the exterior of the booth 108 to determine whether the booth 108 is occupied. This may assist with ensuring that any pharmacy transactions and/or discussions with the booth 108 remain confidential by reducing the likelihood that any individuals within the booth 108 are interrupted by another individual attempting to enter the booth 108.

A desk 120 and chairs 121 and 122 are in the booth 108. The desk 120 itself does not function as a barrier between the customer area 102 and the dispensing area 106. However, alternative embodiment may include, in the booth 108, a barrier between the customer area 102 and the dispensing area 106. Further, a pharmacy computer 123 and a payment device 124 are on the desk 120 and in the booth 108. The payment device 124 may include a credit-card-transaction device, a debit-card-transaction device, a cash till, a point-of-sale (POS) device, one or more other payment devices, or a combination of two or more thereof.

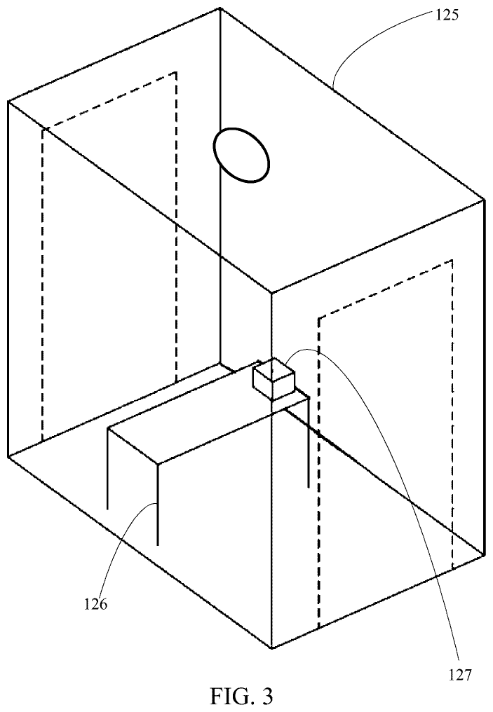

Alternative embodiments may differ. For example, referring to FIG. 3, a booth according to another embodiment is shown generally at 125. A desk 126 is in the booth 125, but no chairs are in the booth 125. As in the embodiment of FIG. 2, the desk 126 itself does not function as a barrier between a customer area and a dispensing area, but alternative embodiments may differ. As a result, in the embodiment shown, the desk 126 is higher than the desk 120 and has a height suitable for an interaction between a pharmacist, pharmacy technician, or other pharmacy staff and a customer when standing. However, alternative embodiments may vary and may have one or more different desks, which may have different heights or which may differ in other ways. A pharmacy computer 127 is on the desk 126 and in the booth 125, and the pharmacy computer 127 may be similar to the pharmacy computer 123. In general, the booth 125 may be similar to the booths 108 and 109, and one or more payment devices such as the payment device 124 may be in the booth 125.

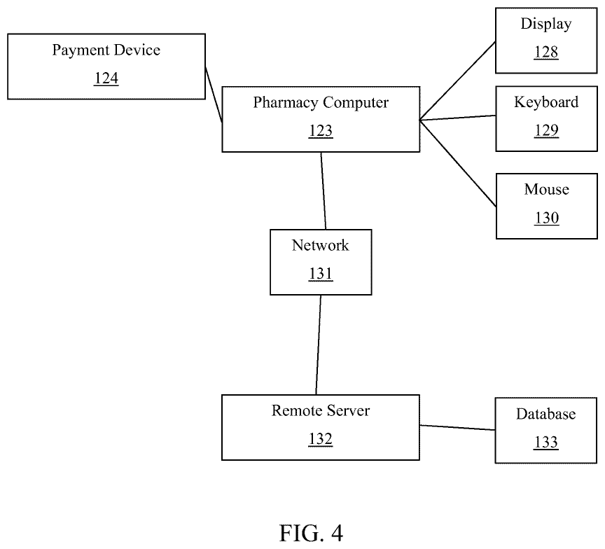

Referring to FIG. 4, the pharmacy computer 123 is in communication with one or more input/output devices, such as a display 128, a keyboard 129, and a mouse 130, which may facilicate interaction between a pharmacist, pharmacy technician, or other pharmacy staff and the pharmacy computer 123 as described herein, for example. Further, the pharmacy computer 123 is in communication with the payment device 124 to facilitate coordination of payments with customer medication records. However, in alternative embodiments, the pharmacy computer 123 need not be in communication with the payment device 124. Also, using a computer network 131, the pharmacy computer 123 is in communication with a remote server computer 132, which includes or is in communication with a database 133. The database 133 may store various records, such as customer medication records. Therefore, the pharmacy computer 123 is configured to access at least a database of customer medication records.

However, FIG. 4 is an example only, and alternative embodiments may differ. For example, alternative embodiments may include one or more different or additional devices in the booth 108. In general, one or more devices in the booth 108 may facilitate one or more pharmacy transactions in the booth 108 as described herein for example. Further, alternative embodiments may include one or more alternatives to the remote server computer 132, to the database 133, or both.

In general, the booth 108 may be enclosed or substantially enclosed on all sides with materials such that the booth 108 is soundproof. For example, the booth 108 may have a sound transmission class (STC) rating of at least 40, at least 45, at least 50, at least 55, or at least 60, although alternative embodiments may differ. More generally, in some embodiments, the booth 108 may be sufficiently soundproof to permit confidential discussions in the booth 108. Additionally or alternatively, the booth 108 may include such translucent materials, opaque materials, or a combination of both such that the booth 108 sufficiently diffuses light exiting, or sufficiently blocks light from exiting, the booth 108 to permit confidential transactions (which may include, for example, collection of personal information, disclosure of personal information, or both) in the booth 108 with auditory privacy, visual privacy, or both. Therefore, the booth 108 may be a location for a confidential pharmacy transaction may therefore be referred to as a “confidentiality booth”.

The booth 109 may be similar to the booth 108, and a pharmacy computer 134 (which may be similar to the pharmacy computer 123) and a payment device (which may be similar to the payment device 124) may be in the booth 109.

In some embodiments, the booth 108 may be designated or used typically for prescription drop-off and may for example be at a prescription-drop-off location in the pharmacy 100. The booth 109 may be designated or used typically for prescription pick-up and may for example be at a prescription-pick-up location in the pharmacy 100.

However, alternative embodiments may differ. For example, if the booth 108 is designated or used typically for prescription drop-off, then the payment device 124 may be omitted, and the booth 109 may include a payment device (which may be similar to the payment device 124) instead.

The pharmacy computer 123, the payment device 124, the pharmacy computer 127, the pharmacy computer 134, one or more other devices, or a combination of two or more thereof may facilitate such pharmacy transactions. For example, during a prescription-drop-off pharmacy transaction in the booth 108, a pharmacist, a pharmacy technician, one or more other pharmacy staff, or a combination of two or more thereof may receive a prescription from a customer, discuss a medical condition that may relate to the prescription, receive personal information from the customer, use the pharmacy computer 123 to cause such personal information or information related to the prescription to be stored in the database 133, cause the pharmacy computer 123 to access the database 133, provide pharmaceutical advice to the pharmacy customer, conduct one or more other activities of a pharmacy transaction, or conduct a combination of two or more thereof, all in relative privacy when compared to such a transaction outside of the booth 108.

As another example, during prescription-pick-up pharmacy transaction in the booth 109, a pharmacist, a pharmacy technician, one or more other pharmacy staff, or a combination of two or more thereof may provide one or more medications to a customer, discuss a medical condition that may relate to the one or more medications, discuss the one or more medications, discuss use of the one or more medications, provide pharmaceutical advice to the pharmacy customer, receive a payment for the one or more medications using the payment device (which may be similar to the payment device 124) in the booth 109, conduct one or more other activities of a pharmacy transaction, or conduct a combination of two or more thereof, all in relative privacy when compared to such a transaction outside of the booth 109.

Therefore, the pharmacy computer 123 and the payment device 124 are examples of pharmacy-transaction devices in the booth 108. In general, the pharmacy computer 123, the payment device 124, one or more other pharmacy-devices, or a combination of two or more thereof may facilitate pharmacy transactions such as those described above.

One or more ventilation systems, such as one or more air pumps for example, may provide air ventilation to the booth 108, to the booth 109, or to both.

As shown in FIG. 1, the booths 108 and 109 are positioned in the pharmacy 100 at central locations that may be typically used for common pharmacy transactions such as prescription drop-off and pick-up. For example, positioning the booths 108 and 109 in the pharmacy 100 adjacent the pharmacy counter 101, integrated into the pharmacy counter 101, attached directly or indirectly to the pharmacy counter 101, with portions of the pharmacy counter 101 on one or both sides of the booths 108 and 109, as described above, may conveniently position the booths 108 and 109 in the pharmacy 100 at central locations that may be typically used for common pharmacy transactions such as prescription drop-off and pick-up.

However, in the embodiment shown, the booths 108 and 109 are not required for pharmacy transactions. A pharmacy computer 135 (which may be similar to the pharmacy computer 123) is also on the pharmacy counter 101 outside of the booths 108 and 109 and may facilitate transactions that do not require privacy.

Some pharmacies, such as the pharmacy 100, may include a separate confidential conselling room in addition to one or more booths such as the booths 108 and 109. Such a separate confidential conselling room may be suitable for longer interactions, such as interactions longer than five or ten minutes for example, and may be designed to obscure view between inside and outside of the separate confidential conselling room. On the other hand, one or more booths such as the booths 108 and 109 may be suitable for more-routine pharmacy transactions, such as prescription pick-up and drop-off for example, and may be suitable for a pharmacist, pharmacy technician, or other pharmacy staff to conduct a pharmacy transaction while working alone and therefore maintaining visibility to at least substantially all of the customer area 102.

The pharmacy 100 is an example only, and alternative embodiments may differ. For example, a pharmacy of an alternative embodiment may have a different configuration, more or fewer pharmacy counters, and more, fewer, or different pharmacy fixtures or structures.

Nevertheless, embodiments such as those described herein may better facilitate greater privacy for pharmacy transactions when compared to other pharmacies.

Although specific embodiments have been described and illustrated, such embodiments should be considered illustrative only and not as limiting the invention as construed according to the accompanying claims.

1. A pharmacy-transaction apparatus comprising at least one booth, each booth of the at least one booth accessible from at least a customer side of a pharmacy counter and a dispensing side of the pharmacy counter opposite the customer side of the pharmacy counter, wherein, at least, at least one pharmacy-transaction device is in one or more of the at least one booth.

2. The apparatus of claim 1 wherein one or more of the at least one booth is adjacent the pharmacy counter.

3. The apparatus of claim 1 or 2 wherein one or more of the at least one booth is integrated into the pharmacy counter. 4 The apparatus of claim 1, 2, or 3 wherein one or more of the at least one booth is attached to the pharmacy counter.

5. The apparatus of claim 1, 2, or 3 wherein one or more of the at least one booth is attached directly to the pharmacy counter.

6. The apparatus of any one of claims 1 to 5 wherein at least portions of the pharmacy counter are on opposite sides of one or more of the at least one booth.

7. The apparatus of any one of claims 1 to 6 further comprising the pharmacy counter. 8 The apparatus of any one of claims 1 to 7 wherein the pharmacy counter comprises retail fixtures for displaying pharmacy products on the customer side of the pharmacy counter.

9. The apparatus of any one of claims 1 to 7 wherein the pharmacy counter comprises retail fixtures displaying pharmacy products on the customer side of the pharmacy counter.

10. The apparatus of claim 8 or 9 wherein at least some of the retail fixtures are on opposite sides of one or more of the at least one booth.

11. The apparatus of claim 8, 9, or 10 wherein at least some of the retail fixtures are shelves.

12. The apparatus of claim 8, 9, 10, or 11 wherein at least some of the retail fixtures are racks.

13. The apparatus of any one of claims 1 to 12 wherein the pharmacy counter comprises product-storage fixtures for storing pharmacy products on the dispensing side of the pharmacy counter.

14. The apparatus of any one of claims 1 to 12 wherein the pharmacy counter comprises product-storage fixtures storing pharmacy products on the dispensing side of the pharmacy counter.

15. The apparatus of claim 13 or 14 wherein at least some of the product-storage fixtures are on opposite sides of one or more of the at least one booth.

16. The apparatus of claim 13, 14, or 15 wherein at least some of the product-storage fixtures are shelves.

17. The apparatus of claim 13, 14, 15, or 16 wherein at least some of the product-storage fixtures are racks.

18. The apparatus of any one of claims 1 to 17 wherein each booth of the at least one booth is substantially enclosed on all sides.

19. The apparatus of any one of claims 1 to 17 wherein each booth of the at least one booth is enclosed on all sides.

20. The apparatus of any one of claims 1 to 19 further comprising the at least one pharmacy-transaction device.

21. The apparatus of any one of claims 1 to 20 wherein the at least one pharmacy-transaction device comprises at least one payment device. 22 The apparatus of any one of claims 1 to 21 wherein the at least one pharmacy-transaction device comprises at least one pharmacy computer.

23. The apparatus of claim 22 wherein the at least one pharmacy computer is configured to access at least a database of customer medication records.

24. The apparatus of any one of claims 1 to 23 wherein one or more of the at least one booth comprises at least one translucent exterior wall.

25. The apparatus of any one of claims 1 to 23 wherein one or more of the at least one booth comprises at least one translucent exterior wall on each side of the booth.

26. The apparatus of claim 24 or 25 wherein the at least one translucent exterior wall permits visibility from within the one or more of the at least one booth to at least substantially all of a customer area on the customer side of the pharmacy counter.

27. The apparatus of any one of claims 1 to 26 further comprising at least one air pump in communication with an interior of one or more of the at least one booth and with an exterior of the at least one booth, the at least one air pump operable to exchange air between the interior of the one or more of the at least one booth and the exterior of the at least one booth.

28. The apparatus of any one of claims 1 to 27 further comprising lighting associated with one or more of the at least one booth operable to illuminate an interior of the one or more of the at least one booth. 29 The apparatus of any one of claims 1 to 28 wherein one or more of the at least one booth is wheelchair-accessible from at least the customer side of the pharmacy counter.

30. The apparatus of any one of claims 1 to 29 wherein one or more of the at least one booth is wheelchair-accessible from at least the dispensing side of the pharmacy counter.

31. The apparatus of any one of claims 1 to 30 wherein one or more of the at least one booth has a sound transmission class (STC) rating of at least 40.

32. The apparatus of any one of claims 1 to 30 wherein one or more of the at least one booth has an STC rating of at least 45.

33. The apparatus of any one of claims 1 to 30 wherein one or more of the at least one booth has an STC rating of at least 50.

34. The apparatus of any one of claims 1 to 30 wherein one or more of the at least one booth has an STC rating of at least 55.

35. The apparatus of any one of claims 1 to 30 wherein one or more of the at least one booth has an STC rating of at least 60.

36. The apparatus of any one of claims 1 to 35 wherein the at least one booth comprises a first booth and a second booth.

37. The apparatus of any one of claims 1 to 36 wherein each booth of the at least one booth is sufficiently soundproof to permit confidential discussions in each booth of the at least one booth with at least auditory privacy.

38. The apparatus of any one of claims 1 to 37 wherein each booth of the at least one booth is sufficiently soundproof to permit collection or disclosure of personal information in each booth of the at least one booth with at least auditory privacy.

39. The apparatus of any one of claims 1 to 38 wherein each booth of the at least one booth sufficiently diffuses light exiting, or sufficiently blocks light from exiting, each booth of the at least one booth to permit confidential transactions in each booth of the at least one booth with at least visual privacy.

40. The apparatus of any one of claims 1 to 39 wherein each booth of the at least one booth sufficiently diffuses light exiting, or sufficiently blocks light from exiting, each booth of the at least one booth to permit confidential transactions in each booth of the at least one booth with at least visual privacy.

41. A method of conducting at least one pharmacy transaction with a pharmacy customer, the method comprising conducting the at least one pharmacy transaction in one or more of the at least one booth of the apparatus of any one of claims 1 to 40.

42. The method of claim 41 wherein the at least one pharmacy transaction is at least one confidential pharmacy transaction.

43. The method of claim 41 or 42 wherein the at least one pharmacy transaction comprises receiving at least one prescription from the pharmacy customer.

44. The method of claim 41, 42, or 43 wherein the at least one pharmacy transaction comprises providing at least one medication to the pharmacy customer.

45. The method of claim 41 or 42, when dependent from claim 36, wherein the at least one pharmacy transaction comprises:
receiving at least one prescription from the pharmacy customer in the first booth; and
providing at least one medication to the pharmacy customer in the second booth.

46. The method of any one of claims 41 to 45 wherein the at least one pharmacy transaction comprises receiving a payment from the pharmacy customer.

47. The method of claim 46, when dependent from claim 21, wherein receiving the payment from the pharmacy customer comprises receiving the payment from the pharmacy customer using the at least one payment device.

48. The method of any one of claims 41 to 47 wherein the at least one pharmacy transaction comprises providing pharmaceutical advice to the pharmacy customer.

49. The method of any one of claims 41 to 48, when dependent from claim 23, wherein the at least one pharmacy transaction comprises causing the at least one pharmacy computer to access at least the database of customer medication records.

50. The method of any one of claims 41 to 49 wherein the at least one pharmacy transaction comprises receiving personal information from the pharmacy customer.

51. The method of claim 50, when dependent from claim 23, wherein the at least one pharmacy transaction comprises causing the at least one pharmacy computer to cause at least the database of customer medication records to store at least some of the personal information.
